(19) United States
(12) Patent Application Publication
(10) Pub. No.: US 2023/0310197 A1
(43) Pub. Date: Oct. 5, 2023

(54) Device for Supporting the Spine of a User and Improving Posture

(71) Applicant: Colm Campbell, Dublin 24 (IE)

(72) Inventor: Colm Campbell, Dublin 24 (IE)

(21) Appl. No.: 18/194,618

(22) Filed: Mar. 31, 2023

(30) Foreign Application Priority Data
Apr. 1, 2022 (IE) S2022/0059

(51) Int. Cl. A61F 5/02

(52) U.S. Cl. CPC A61F 5/026

(57) ABSTRACT

A device (10) for improving posture and for supporting the spine of a user. The device (10) comprising a main spine bar (1) arranged to be secured to the back of the user at the location of the spine by a strap arrangement. The strap arrangement comprising a waist strap belt (2) connected to a proximal lower end of the spine bar (10) and comprising a first and a second member configured to wrap around the waist of the user and connect by first connecting means. The strap arrangement further comprising shoulder and torso straps (3) connected at proximal upper end of the spine bar (1) and configured to wrap around the shoulders and torso of the user and connect together by second connecting means, so that when the shoulder and torso straps (3) have connected at a predetermined location the shoulders of the user are uniformly pulled backwards thereby causing the spine of the user to assume an S-shape posture form.

The present invention relates to a device for improving the posture of a user, a moulding device for shaping the spine bar of the device to the unique S shape curvature of the user and a method for manufacturing the device.

### BACKGROUND TO THE INVENTION

Back pain is a common cause of discomfort among people of all ages and is associated with poor posture. The positive effects of good posture are well documented through extensive medical studies. These medical studies have shown that when good posture is assumed, the body weight is evenly distributed over the spine and lower joints, resulting in the uniform distribution of pressure on the intervertebral discs of the spine. Ideally, in a body exhibiting proper posture, the spine, when viewed from the side, the spine forms a slight S-shape with the shoulders pulled back.

Proper posture should be maintained during both static and dynamic activities. Unfortunately, our increasingly inactive lifestyle, wherein many hours are spent sitting in front of a computer, driving a car, or watching television, hurts posture.

Furthermore, in most cases, it is difficult to maintain awareness of our posture, especially during challenging activities, such as running, working on a computer, and the like.

There is a wide range of devices available for improving posture. These devices range from specialised chairs that promote good posture while sitting, to garments that can be worn by the user to promote good posture during dynamic activities. However, such devices are very specialised and difficult to incorporate into the lifestyle of the user. For example, known garments for improving posture they may be constructed from heavy materials, and can often only be worn for a limited period. Furthermore, such garments can be quite bulky making difficult to conceal them under clothing.

Therefore, there is a need for developing an improved device that helps the user to assume good posture during static and dynamic activities.

### SUMMARY OF THE INVENTION

It is an object of the present invention to provide an improved device for supporting the spine of the user and promoting good posture that overcomes the disadvantages of the prior art.

This object is achieved according to a first aspect of the present invention. The present invention provides a device for improving posture and for supporting the spine of a user, the device comprising a main spine bar arranged to be secured to the back of the user at the location of the spine by a strap arrangement, the strap arrangement comprising a waist strap connected to a proximal lower end of the spine bar and comprising a first and a second member configured to wrap around the waist of the user and connect by first connecting means, and shoulder and torso straps connected at proximal upper end of the spine bar and configured to wrap around the shoulders and torso of the user and connect by second connecting means, so that when the shoulder and torso straps have connected the shoulders of the user are uniformly pulled backwards thereby causing the spine of the user to assume an S-shape posture form.

It has been found that the combination of the spine bar with the strapping arrangement has the advantageous effect of correctly supporting the spine to maintain an S-shape form during use and thus improve the posture of the user. Furthermore, the device has the advantage that it can be used during static and dynamic activities over long periods without any adjustments from the user.

According to embodiments of the present invention, the spine bar is made of from a tough, rigid mouldable light-weight material. For example, the spine bar may be made out of a thermoplastic polymer, e.g. polycarbonate, polyester based polymers, e.g. polycaprolactone, Teflon, polyethene, and the like. Furthermore, the spine bar may be made from a metal-based material, e.g. aluminium, rubber-based material, wood or any other material that can be shaped and offers enough resistance so as to correctly support the spine of the user to maintain an S-shape form during use.

It has been found that by manufacturing the spine bar from a tough, rigid mouldable light-weight material has the advantage of providing better spine support and at the same time being compact and comfortable to the user. Also, due to the reduced size of the spine bar, the device can be discretely worn under regular clothing.

According to embodiments of the present invention, the spine bar is shaped according to the curvature of the user's spine when in perfect posture.

It has been found that by shaping the spine bar to match the individual user's spine curvature when in perfect posture, has the effect of improving comfort to the user while further improving the support provided to the spine.

According to embodiments of the present invention, the spine bar extends from the upper thoracic vertebrae to the bottom lumbar vertebrae.

It has been found that by providing the spine bar between the upper thoracic and lower lumbar vertebrae has the advantage of that the spine is supported along its length, which ensures that the S-shape of the spine is maintained over a wide range of motions and activities.

According to embodiments of the present invention, the first and second connection means for connecting the waist belt members, and the shoulder and torso straps may comprise Velcro strips, tensioners, buckle or clasps and the like.

According to embodiments of the present invention, waist belt members, and the shoulder and torso strips are made from an elastic material.

According to a second aspect of the present invention, an adjustable moulding device is provided for shaping the spine bar to the S shape of the individual user spine. The moulding device comprises a fixed member plate connected to a heat resistance surface, and a movable member plate configured to move in a relative direction to the fixed member plate. The fixed and movable member plates comprise securing elements for securing respectively a positive and a negative moulding element. The positive moulding element defines the positive shape of the spine of the user, while the negative moulding element defines the corresponding negative shape of the spine of the user and precisely matches the shape of the positive moulding element. According to embodiments of the present invention, the positive and negative moulding elements are secured to the respective fixed and movable member plates by positioning on top of each element a securing member plate. The securing member plate is clamped into position such that the respective moulding elements are secured to the respective member plates. According to embodiments of the present invention, the fixed and movable member plates are made of a tough, rigid material such as polycarbonate, metal and the like. Similarly, the securing member plate is made from a tough, rigid material such as polycarbonate, metal and the like. The moulding device is configured for shaping the spine bar by applying pressure to a strip of material positioned between the positive and the negative moulding elements. The material strip may be heated to a mouldable temperature prior or during its compression.

According to embodiments of the present invention, the moulding device instead of or in addition to the securing member plate may be provided with grooves on each of the fixed and movable members, which can be used to secure the positive and negative moulding elements.

According to a third aspect of the present invention, a method of manufacturing the spine bar is provided. The method comprises the steps of:
a) Obtaining the unique S shape of the user's spine;
b) Marking the obtained S shape down the middle of a flat material, wherein the length of the flat material corresponds to the actual length of the user's spine measured between the upper thoracic vertebrae to the bottom lumbar vertebrae;
c) Cutting the flat material along the marked S shape, so as to obtain two pieces, a positive half portraying the positive shape of user's spine and a negative half portraying the negative shape of the user's spine, wherein the positive half defines the positive moulding element while the negative half defines the negative moulding element;
d) Providing a moulding device including the positive half and negative half moulding elements;
e) Securing the positive moulding element on the fixed member plate of the moulding device and the negative moulding element on the movable member plate by clamping a securing member plate on top of the moulding elements, wherein the moulding elements are secured such that the outline of the negative and positive shape of the user's spine is clearly visible;
f) Positioning a strip of heated material having the desired dimensions between the first and second moulding elements; and
g) Applying pressure on the strip of heated material by moving the movable member plate relative to the fixed member such that the strip of material is compressed by the positive and negative moulding elements; and
h) Continue applying pressure until the material strip takes the desired shape.

According to embodiments of the present invention, the unique S shape of the user's spine may be obtained by any suitable means, which may include but not limited to the direct measurement of the S shape using a spine curvature measuring device, by a computer scanning method using a specific software and hardware modules, from a photograph, and the like.

### BRIEF DESCRIPTION OF THE DRAWINGS

The following drawings are provided as an example of an inventive Embodiment to explain further and describe various aspects of the invention.

FIG. 1 shows an example of a device according to the embodiments of the present invention.

FIGS. 2a to 2c show different views of the exemplified device of FIG. 1 as worn by the user.

### DETAILED DESCRIPTION

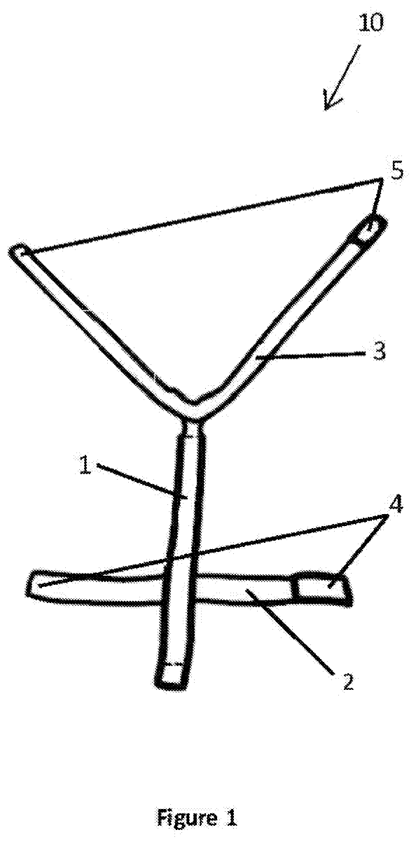

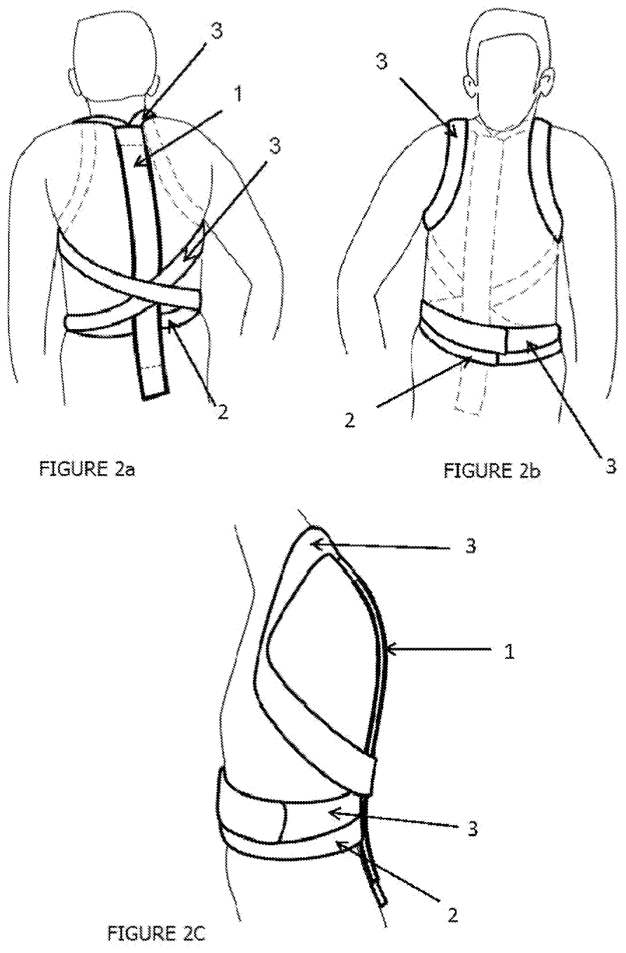

The present invention will be illustrated using the exemplified embodiments shown in the FIGS. 1 and 2, which will be described in more details below. It should be noted that any references made to dimensions are only indicative and do not restrict the invention in any way. While this invention has been particularly shown and described with reference to certain illustrated embodiments thereof, it will be understood by those skilled in the art that various changes in form and details may be made therein without departing from the scope of the invention encompassed by the appended claims.

FIG. 1 shows an example of a device 10 according to the present invention. The device is provided with a spine bar 1 configured to be secured to the back of the user at the location of the spine by a strap arrangement. The strap arrangement is provided with a waist belt 2 comprising a first and a second member arranged to wrap around the waist of the user and secured in place by a first connecting means 4. The waist belt 2 is connected at a proximal lower end of the spine bar 1 and is arranged when connected to secure the spine bar at the lumbar section of the spine. The strap arrangement is further provided with a shoulder and torso straps 3, connected to at a proximal upper end of the spine bar 3 and are arranged to come over the shoulders and then wrap around the under arms and then the torso such that when connected by the second connecting means the shoulders are pulled backwards, thereby forcing the spine of the user to assume the correct posture. The waist belt 2, and the shoulder and torso straps 3 may be made from an elastic strap material that is arranged when a pulling force is applied to one of its ends to stretch by a predetermined length. Once the forces are no longer applied, the elastic strap material returns to its original shape. The elastic strap material may be in the form of stretchable fabric made from synthetic fabric which stretches when a force is applied at one its ends. The spine bar 1 may be made from a tough, rigid mouldable material, and may be covered by a soft fabric to feel comfortable when secured on the back of the user. The soft fabric may be the same stretchable fabric used for the straps 2 and 3, but other materials may be used that are known in the art to enhance comfort when in contact with the skin of the user. The first and second connection means 4 and 5 for connecting the waist belt members 2, and the shoulder and torso straps 3 may comprise Velcro strips, tensioners, buckle or clasps and the like.

The spine bar 1 may be made of from a tough, rigid mouldable light-weight material. For example, the spine bar may be made out of a thermoplastic polymer, e.g. polycarbonate, polyester based polymers, e.g. polycaprolactone, Teflon, polyethene, and the like. Furthermore, the spine bar 1 may be made from a metal-based material, e.g. aluminium, rubber-based material, wood or any other material that may be shaped so as to support the spine of the user. The spine bar 1 may be shaped according to the curvature of the user's spine when in perfect posture. In this way, the spine bar 1 conforms to the contours of the user's spine thereby offering better support to the spine when worn. The spine bar 1 may be designed to support a specific region of the user's spine depending on the needs of the user. For example, the spine bar 1 may extend from the upper thoracic vertebrae to the bottom lumbar vertebrae.

For example, the device 10 may be made from a tough, rigid, lightweight material moulded to the precise shape of the individual spine and held in position by a waist belt 2, of approximately 2 inches (around 50.0 mm.) and two elasticised shoulder and torso straps 3. The total weight of the device is approximately ½ lb., around 350 grams. The width of the spine bar 1 is 2 inches (around 50.0 mm), and ⅕ inch (5 mm) thick. The length of the spine bar 1 varies according to the length of the user's spine. The device is concealed under clothing, and because it is light and narrow, the only part of the body it makes contact with is the spine, the person wearing it is unaware of its presence. As a result, the device 10 can be worn for long periods during both static and dynamic activities.

The spine bar 1 is shaped according to the spine curvature of the user, when in a good posture positioned, by means of a moulding machine. First, the shape of the user's spine is obtained. This can be done by direct measurement of the user's spine curvature via specialised spine measuring device, by means of an electronic scanning method, or by means of a photographic process. Then the obtained shape is marked on a flat material, such as a white display board, having predetermined dimensions. For example, the flat material may measure 800 mm×150 mm with the spinal shape drawn down the middle. The flat material is cut along the spinal shape, resulting in two pieces of flat material, each measuring 800 mm×75 mm. One side could be called the negative moulding element, the other the positive moulding element. The positive and the negative moulding elements are secured in a moulding device, which consists of a 25 mm plate, e.g. made from polycarbonate, metal and the like, that measures 800 mm×75 mm, which is fixed to a heat-resistant surface. On top of this plate a securing member plate, e.g. made from the same material as the fixed member plate, with the same dimensions is placed. The positive moulding element is sandwiched between the two plates. The positive moulding element is then adjusted so that the containing the positive shape of the spine is showing. The securing member plate is then clamped to the fixed plate, and the moulding element is secured into the desired position. The negative moulding element is secured in a similar manner. A separate securing plate is clamped to a member plate that is movable in a relative direction to the fixed member plate, e.g. may be configured to move along a left to right track. Moving this movable member plate allows the positive and negative spinal shapes to mate. The spine bar 1 is made by positioning a heated strip of material with the predetermined dimensions between the positive and negative moulding elements. For example, the strip material may have a thickness of 5 mm, a width of 50.0 mm, while the length is dependent on the length of the user's spine. The edge of the heated strip of material is then placed down on the heat resistant surface with the side vertical and in contact with the positive. The movable member plate containing the negative shape is then pressed against the pliable polycarbonate until it deforms into the precise shape of the spine. Pressure continues to be applied until the shape becomes rigid.

FIGS. 2a and 2c show different views of the device 10 as worn by the user. FIG. 2a shows a back view of the device 10 as worn by the user. As shown in the figures the device is positioned on the back of the user such that the spine bar 1 is secured by the strap arrangement along the spine of the user. The waist belt members 2, are wrapped around the waist of the user and connected to the front of the user's waist by Velcro strips or other means as previously discussed. The shoulder and torso straps 3 come over the shoulders, then wrap around the under the arms and then the torso. They overlap at the back, as shown in FIG. 2a, and connected to the front of the user as shown in FIG. 2b, by Velcro strips or other connecting means as previously discussed. With the shoulder and torso straps wrapped around the shoulders and torso, the shoulders are uniformly pulled backwards thereby putting the body in good posture form.

It is to be understood that the invention is not limited to the specific details described herein which are given by way of example only and that various modifications and alternations are possible without departing from the scope of the invention as defined in the appended claims.

1. A device for improving posture and for supporting the spine of a user, wherein the user has a torso with shoulders, a back, a spine and a waist, the device comprising:
a main spine bar having a proximal lower end and an opposite proximal upper end, wherein the main spine bar is secured to the back of the user at the location of the spine by a strap arrangement, the strap arrangement comprising
a waist strap connected to the proximal lower end of the spine bar and comprising a first and a second member configured to wrap around the waist of the user and connect by first connecting means, and
shoulder and torso straps connected at the proximal upper end of the spine bar and configured to wrap around the shoulders and torso of the user and connect by second connecting means, so that when the shoulder and torso straps have connected the shoulders of the user are uniformly pulled backwards thereby causing the spine of the user to assume an S-shape posture form.

2. A device according to claim 1, wherein the spine has an upper thoracic vertebrae and a bottom lumbar vertebrae, and wherein the spine bar extends, when in contact with the spine of the user, from the upper thoracic vertebrae to the bottom lumbar vertebrae.

3. A device according to claim 1, wherein the spine has a curvature and wherein the spine bar is shaped according to the curvature when in a perfect posture.

4. An adjustable moulding device for shaping a spine bar to the S shape of a spine, the moulding device comprising:
a fixed member plate connected to a heat resistance surface, and
a movable member plate configured to move in a relative direction to the fixed member plate, wherein the fixed member plate and the movable member plate comprise securing elements for securing a positive moulding element and a negative moulding element respectively;
wherein the positive moulding element defines the positive shape of the spine, while the negative moulding element defines the corresponding negative shape of the spine and precisely matches the positive moulding element,
the positive moulding element and the negative moulding element being secured to the respective fixed member plate and the movable member plate by positioning on top of the fixed member plate and the movable member plate a securing member plate, which is clamped into position such that the positive moulding element and the negative moulding element are respectively secured to the fixed member plate and the movable member plate;
wherein the moulding device is configured for shaping the spine bar by applying pressure to a strip of material positioned between the positive moulding element and the negative moulding element.

5. A method of manufacturing a spine bar comprising the steps of:
a) obtaining a unique S shape of the user's spine;
b) marking the obtained S shape down the middle of a flat material, wherein the length of the flat material corresponds to the actual length of the user's spine measured between the upper thoracic vertebrae to the bottom lumbar vertebrae;
c) cutting the flat material along the marked S shape, so as to obtain two pieces, a positive half portraying the positive shape of user's spine and a negative half portraying the negative shape of the user's spine, wherein the positive half defines the positive moulding element while the negative half defines the negative moulding element;
d) providing a moulding device including the positive half and the negative half;
e) securing the positive moulding element on the fixed member plate of the moulding device and the negative moulding element on the movable member plate by clamping a securing member plate on top of the positive moulding element and the negative moulding element, wherein the positive moulding element and the negative moulding element are secured such that an outline of the negative and positive shape of the user's spine is clearly visible;
f) positioning a strip of heated material between the positive moulding element and the negative moulding element;
g) applying pressure on the strip of heated material by moving the movable member plate relative to the fixed member plate such that the strip of material is compressed by the positive moulding element and the negative moulding element; and
h) continue applying pressure until the material strip takes a desired shape.
